(19) United States
(12) Patent Application Publication
(10) Pub. No.: US 2025/0169757 A1
(43) Pub. Date: May 29, 2025

(54) WATCH ACCESSORY, WRISTBAND AND USER MONITORING AND MANAGEMENT SYSTEM

(71) Applicant: Shengrong LI, Ningbo City (CN)

(72) Inventor: Shengrong LI, Ningbo City (CN)

(21) Appl. No.: 18/406,392

(22) Filed: Jan. 8, 2024

(30) Foreign Application Priority Data
Nov. 29, 2023 (CN) 202323239937.8

(51) Int. Cl. A61B 5/00; A44C 5/00; A61B 5/021; A61B 5/022; A61B 5/0225; A61B 5/024; G04G 21/02

(52) U.S. Cl. CPC A61B 5/681; A44C 5/0007; A61B 5/0004; A61B 5/0022; A61B 5/02108; A61B 5/02233; A61B 5/0225; A61B 5/746; G04G 21/025; A61B 5/02416; A61B 2560/0214; A61B 2560/0443

(57) ABSTRACT

The present disclosure provides a watch accessory, a wristband and a user monitoring and management system, wherein the watch accessory includes a watchband as well as a circuit board and a blood pressure measuring unit that are arranged on the watchband, the watchband is detachably connected with a watch head, and the blood pressure measuring unit is electrically connected with the circuit board to measure a human pressure blood. Thus, the blood pressure measurement of a user may be implemented without configuring the watch head, to facilitate the use of users, and the users may replace the watch head, to meet the users' demands on different watch heads.

### CROSS-REFERENCE TO RELATED APPLICATIONS

The application claims priority of Chinese patent application CN2023232399378 filed on 2023 Nov. 29, which is incorporated herein by reference in its entireties.

### TECHNICAL FIELD

The present disclosure relates to the technical field of wearing products, and in particular to a watch accessory, a wristband and a user monitoring and management system.

### BACKGROUND

At present, the existing smart watch generally includes a watch head and a watchband connected with the watch head. To achieve blood pressure measurement, generally, an air bag needs to be provided on the watchband, an air pump, a circuit board and a pressure sensors are provided on the watch head, the air pump is controlled to drive through the circuit board in the watch head to achieve inflating and deflating the air bag, the watchband can sense the pressure change of arteries through the pressure sensor and the detected pressure value is transmitted to the circuit board, and the circuit board calculates the blood pressure based on the pressure value. But this way needs to rely on the watch head for implementing, and after the watch head is replaced, the watchband cannot achieve the role of blood pressure measurement.

### SUMMARY

The main objective of the present disclosure is to provide a watch accessory, a wristband and a user monitoring and management system, to solve the problem that the existing smart watch can implement blood pressure measurement with the cooperation of a watch head and a watchband.

To resolve the foregoing technical problem, technical solutions are provided in the present disclosure.

A watch accessory, including:
a watchband detachably connected with a watch head;
a circuit board arranged on the watchband; and
a blood pressure measuring unit arranged on the watchband and electrically connected with the circuit board to measure a human pressure blood.

In one possible embodiment, the circuit board is a flexible circuit board, which is arranged in the watchband.

In one possible embodiment, the blood pressure measuring unit includes a photoelectric sensor that is arranged on the watchband and electrically connected with the circuit board; and
the photoelectric sensor generates a pulse wave electrical signal according to an intensity of a reflected light fed back by a blood fluctuation due to a human heart beat, the pulse wave electrical signal is fed back to the circuit board, and the circuit board calculates a blood pressure value according to the pulse wave electrical signal.

In one possible embodiment, the circuit board is integrated with an MCU (Microprogrammed Control Unit) and an air pump drive circuit electrically connected with the MCU, the blood pressure measuring unit includes an air pump electrically connected with the air pump drive circuit, an air bag connected with the air pump and a pressure sensor electrically connected with the MCU, the air pump is arranged in the watchband, and the air bag is arranged in a length direction of an inner side wall of the watchband; and the air pump drive circuit drives the air pump to inflate and deflate the air bag based on an electrical signal output by the MCU, the air bag applies a pressure to a user wrist when being inflated, the pressure sensor is configured to detect a pressure value when the user's pulse beats, generate a pulse wave electrical signal, and feed back the signal to the MCU; the pressure sensor is further configured to detect a pressure value change during inflation and deflation of the air bag, generate an amplitude change electrical signal and feed back the signal to the MCU; and the MCU calculates the blood pressure value according to the pulse wave electrical signal and the amplitude change electrical signal.

In one possible embodiment, the watch accessory further includes a cell and a wireless charging coil that are electrically connected with the circuit board and arranged in the watchband, and the cell is used for supplying power to the circuit board.

In one possible implementation, the watchband includes a protective housing and a band body connected with the protective housing, and a mounting slot for placing the watch head is formed in the protective housing.

In one possible implementation, the circuit board, the air pump and the cell are arranged on the band body or the protective housing, and the air bag is arranged in a length direction of the band body.

In one possible implementation, the watchband includes the band body, of which both ends are provided with connectors detachably connected with the watch head.

In one possible implementation, the watch accessory further includes a physiological index collecting unit that is electrically connected with the circuit board, the physiological index collecting unit is configured to collect physiological indexes of a wearer in real time or periodically, and the physiological indexes include one or more of a blood pressure value, a heart rate value, blood oxygen, blood glucose, a body temperature, blood lipid, uric acid and a motion numerical value.

In one possible implementation, the watch accessory further includes a wireless transmission unit that is electrically connected with the circuit board and used for communicating with a mobile terminal, the circuit board is configured to upload the blood pressure value, collected by the blood pressure measuring unit, and the physiological indexes, collected by the physiological index collecting unit, to a cloud through the wireless transmission unit.

In one possible implementation, the watch accessory further includes a housing that is separately mounted with the circuit board, the blood pressure measuring unit, the wireless transmission unit, the physiological index collecting unit, the cell and the wireless charging coil; and
the housing is arranged outside the band body, or a groove is formed on or inside the band body, the housing is arranged in the groove and in interference fit with the groove, and the housing for mounting the circuit board is made of soft rubber.

In one possible implementation, a wiring hole that communicates with an interior and is used for wiring is formed in the housing, a plurality of wiring channels are arranged on the band body, and the plurality of wiring channels are electrically connected lead wiring in respective among various components of the circuit board, the blood pressure measuring unit, the wireless transmission unit, the physiological index collecting unit, the cell and the wireless charging coil; and
the blood pressure measuring unit includes the air pump electrically connected with the circuit board, the air bag connected with the air pump and the pressure sensor electrically connected with the circuit board, and the housing used for mounting the air pump is also provided with an air outlet and an air inlet that are connected with the air pump.

In one possible implementation, the physiological index collecting unit includes a fall sensor that is electrically connected with the circuit board and arranged in the band body, and the circuit board judges whether the user falls or not according to an electrical signal fed back by the fall sensor; and
when the circuit board judges that the user falls, position information is uploaded to the cloud through the wireless transmission unit.

In one possible implementation, the watch accessory further includes an alarm module that is electrically connected with the circuit board and arranged on the band body, the alarm module is configured to send an alarm signal when the circuit board judges that the user falls or the physiological indexes exceed a threshold value.

In one possible implementation, the watch accessory further includes a positioning unit that is electrically connected with the circuit board and used for detecting a user position, and the positioning unit is arranged in the band body; and
when the circuit board judges that the user falls, the position information positioned by the positioning unit is uploaded to the terminal through the wireless transmission unit.

In one possible implementation, the physiological index collecting unit includes an acceleration sensor that is electrically connected with the circuit board, the acceleration sensor is arranged in the band body, the circuit board calculates a motion numerical value according to an electrical signal fed back by the acceleration sensor, and the motion numerical value is one or more of a step number, a motion distance and a calorie consumption.

In one possible implementation, the watch accessory further includes a heart rate sensor that is electrically connected with the circuit board and arranged in the band body, and the circuit board calculates a heart rate value according to an electrical signal fed back by the heart rate sensor.

In one possible implementation, the watch accessory further includes a display that is electrically connected with the circuit board, and the display is configured to display the physiological indexes and testing numerical values; and
the band body further includes a switching element that is electrically connected with the circuit board and arranged on the band body, the circuit board is configured to receive an electrical signal through which the user operates the switching element and/or receive an electrical signal for operation at an APP (Application) end, and start a detection function or switch the physiological indexes displayed on the display.

In one possible implementation, the band body is adjusted in length to be suitably used for wrist widths of different users.

In one possible implementation, the band body includes an upper watchband and a lower watchband, each of which has one end detachably connected with the watch head, the air bag is arranged at one end of a lower surface of the lower watchband that is close to the watch head, the air pump and the pressure sensor are arranged on the lower watchband and close to one end of the watch head; both the physiological index collecting unit and the wireless transmission unit are arranged on the circuit board, both the cell and the display screen are located at one end of the lower watchband that is away from the watch head, and the display screen and the air bag are located on the same side; a threading hole is formed in one end of the upper watchband that is close to the watch head, one end of the lower surface of the upper watchband that is away from the watch head is provided with a second block, and a plurality of second card slots extending in a length direction of the watchband are formed at a position where the lower watchband corresponds to the air bag; and when the watchband is worn on a wrist and after the lower watchband passes through the threading hole at one end away from the watch head, the second block is placed in the second card slot that is in the direction close to the watch head, and after folded back and forth at one end away from the watch head, the lower watchband is detachably connected with the upper watchband.

In one possible implementation, a plurality of third card slots extending in the length direction of the watchband are formed in the upper watchband, a third block is arranged at a position where the lower surface of the lower watchband is away from the watch head, and after folded back and forth at one end of the lower watchband, the third block is clamped with the third card slots.

In one possible implementation, after folded back and forth at one end away from the watch head, the lower watchband is fixed together with the upper watchband through magnet adsorption or magic stickers.

In one possible implementation, the lower watchband includes a first watch section detachably connected with the watch head and a second watch section connected with the first watch section, the air bag is located on the first watch section or the second watch section, the circuit board is at least partially located on the first watch section, and a width of the threading hole is greater than that of the second watch section but less than that of the first watch section.

In one possible implementation, the second card slot is in a two-column setting and close to both sides of the first watch section.

In one possible implementation, a storage slot for placing drugs is formed in the watchband, and a notch of the storage slot is provided with a cover body.

According to another aspect, the present disclosure further provides a wristband, including:
a band body having two ends detachably connected;
a circuit board arranged on the band body; and
a blood pressure measuring unit arranged on the band body and connected with the circuit board to measure a human pressure blood.

According to still another aspect, the present disclosure further provides a user monitoring and management system, including:
a watch accessory described above;
a cloud communicating with the watch accessory, where the cloud is configured to receive physiological indexes transmitted by a wireless transmission unit and perform monitoring, and to send a first alarm signal to the wireless transmission unit when the physiological indexes exceed a threshold value; and
the circuit board receives the first alarm signal fed back by the cloud through the wireless transmission unit and sends an alarm through an alarm module.

In one possible implementation, the user monitoring and management system further includes a monitoring terminal that communicates with the cloud;
the cloud is also configured to send a second alarm signal to the monitoring terminal when the physiological indexes exceed the threshold value during a preset time period, and the monitoring terminal receives a reminding signal sent by the second alarm signal; and
the cloud is further configured to transmit an execution measurement signal to the watch accessory during user operation, and the circuit board receives the execution measurement signal through the wireless transmission unit to drive a blood pressure measuring unit to work.

The present disclosure has the following beneficial effects: compared with the prior art, the blood pressure measurement is implemented by configuring the circuit board and the blood pressure measuring unit on the watchband, the user's blood pressure measurement may be implemented without configuring the watch head, to facilitate the use of users, and the users may replace the watch head, to meet the users' demands on different watch heads and solve the problem that the smart watch may implement the blood pressure measurement with the cooperation of the watchband and the watch head in the existing technology.

### BRIEF DESCRIPTION OF THE DRAWINGS

In order to explain the technical solutions of the embodiments of the present disclosure more clearly, the following will briefly introduce the accompanying drawings used in the embodiments. Apparently, the drawings in the following description are only some embodiments of the present disclosure. Those of ordinary skill in the art can obtain other drawings based on these drawings without creative work.

FIG. 1 is a schematic diagram of a watch accessory used in cooperation with a watch head in one embodiment of the present disclosure.

FIG. 2 is a solid diagram of a watch accessory in one embodiment of the present disclosure.

FIG. 3 is an exploded diagram of a watch accessory used in cooperation with a watch head in one embodiment of the present disclosure.

FIG. 4 is a perspective diagram of a band body in a watch accessory in one embodiment of the present disclosure.

FIG. 5 is a structure diagram of a watch accessory in another embodiment of the present disclosure.

FIG. 6 is a structure diagram of a watch accessory in another embodiment of the present disclosure.

FIG. 7 is a functional block diagram of a user monitoring and management system in one embodiment of the present disclosure.

FIG. 8 is a schematic diagram of a watch accessory used in cooperation with a watch head in another embodiment of the present disclosure.

FIG. 9 is a schematic diagram of another view of a watch accessory used in cooperation with a watch head in another embodiment of the present disclosure.

FIG. 10 is a schematic diagram of a watch accessory removed of an air bag and used in cooperation with a watch head in another embodiment of the present disclosure.

FIG. 11 is a state diagram of a wearing process of a watch accessory removed of an air bag in another embodiment of the present disclosure.

FIG. 12 is a schematic diagram of a wristband in one embodiment of the present disclosure.

FIG. 13 is a functional block diagram of a user monitoring and management system in one embodiment of the present disclosure.

### DETAILED DESCRIPTION OF THE EMBODIMENTS

The accompanying drawings in the embodiment of the present disclosure are combined, The technical scheme in the embodiment of the present disclosure is clearly and completely described, Obviously, the described embodiment is only a part of the embodiment of the present disclosure, but not all embodiments are based on the embodiment of the present disclosure, and all other embodiments obtained by ordinary technicians in the field on the premise of not doing creative work belong to the protection range of the present disclosure.

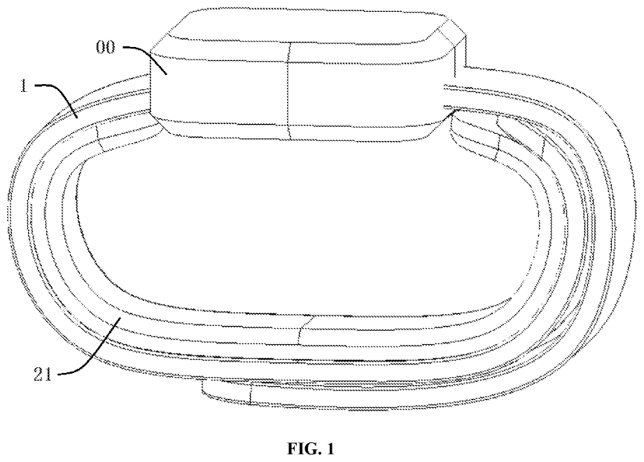

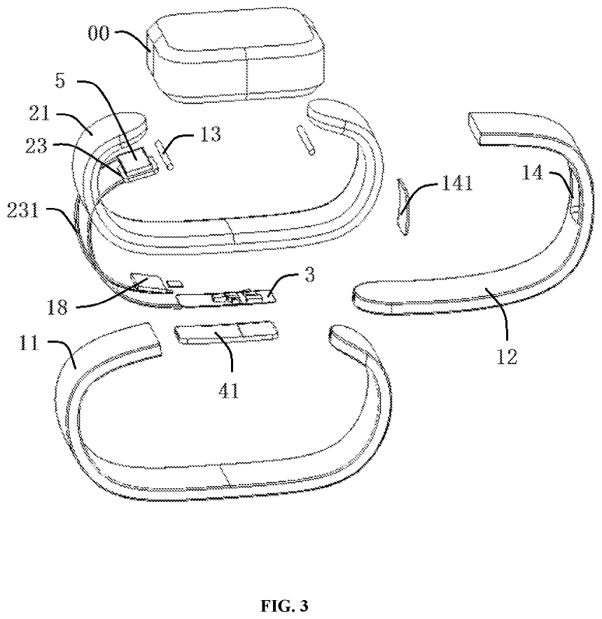

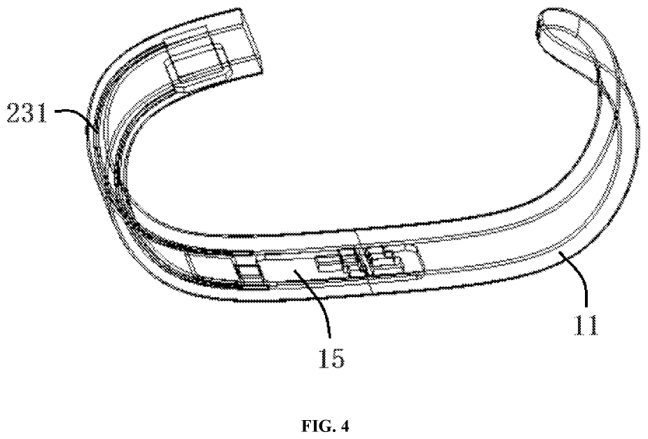

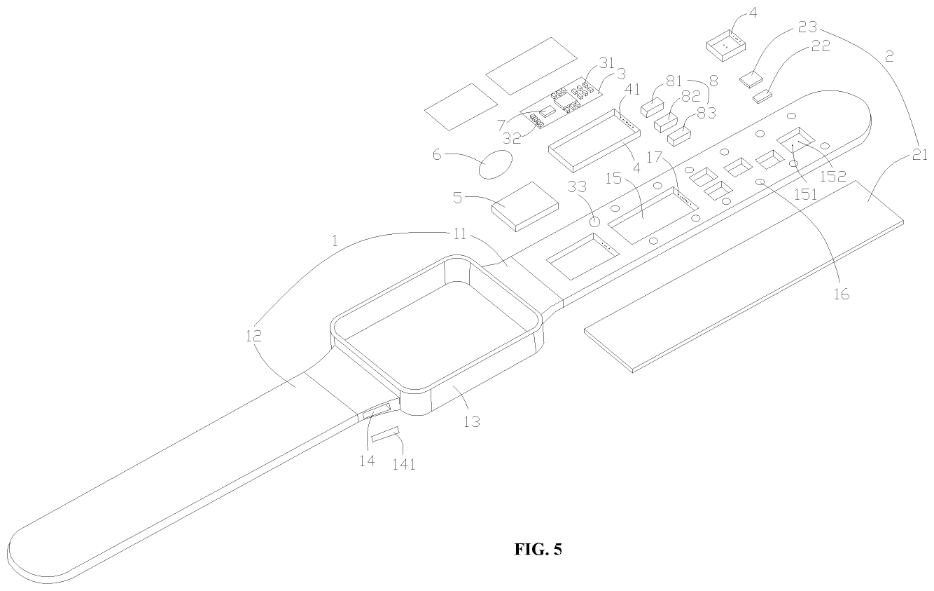

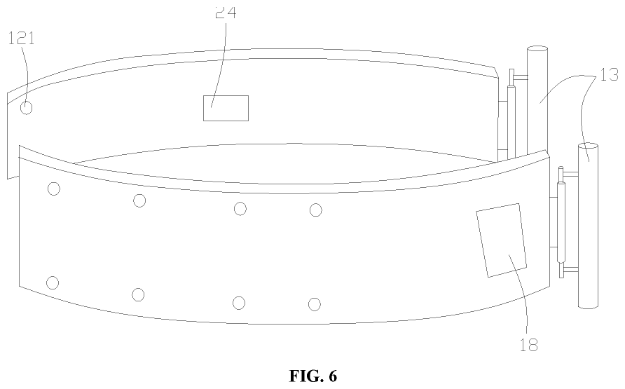

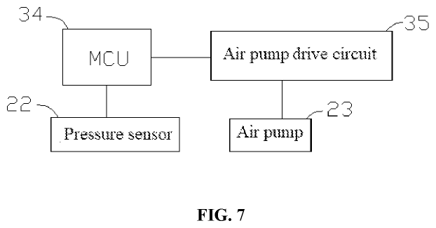

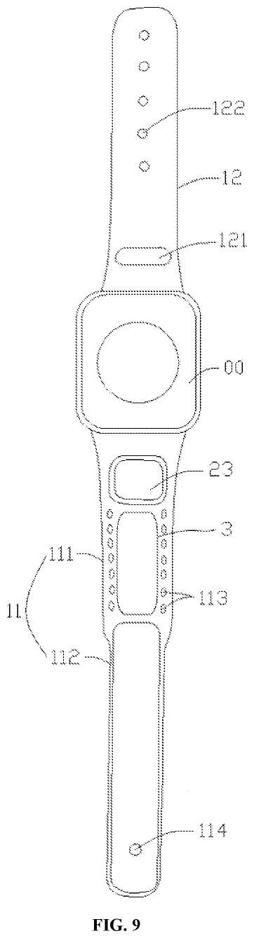

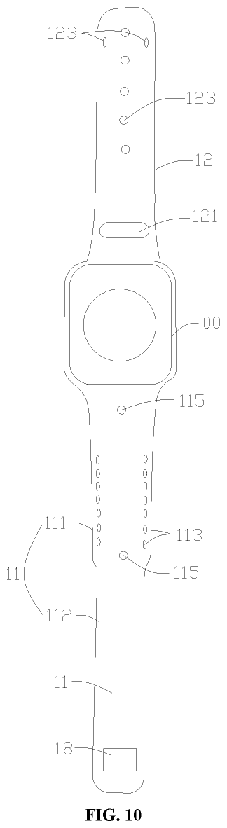

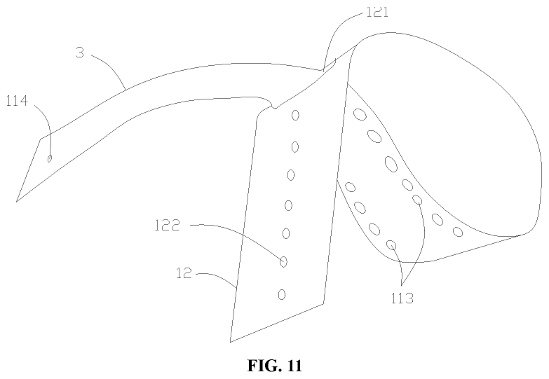

Referring to FIGS. 1-11, which is a watch accessory 10 in embodiments of the present disclosure.

The watch accessory 10 includes a watchband 1, and a circuit board 3 and a blood pressure measuring unit 2 that are arranged on the watchband 1, where the watchband 1 is detachably connected with a watch head 00, and the blood pressure measuring unit 2 is electrically connected with the circuit board 3 to measure a human blood pressure.

In this embodiment, the blood pressure measurement is implemented by configuring the circuit board 3 and the blood pressure measuring unit 2 on the watch accessory 10, the user's blood pressure measurement may be implemented without configuring the watch head 00, to facilitate the use of users, and the users may replace the watch head 00, to meet the users' demands on different watch heads 00 and solve the problem that the smart watch may implement the blood pressure measurement with the cooperation of the watch accessory 10 and the watch head 00 in the existing technology.

In one possible implementation, the circuit board 3 is a flexible circuit board 3, which is arranged in the band body 1. The flexible circuit board 3 has a soft toughness and can be bent to fit the use of the watchband 10, when the watch band 10 is fixed to a user wrist, the circuit board 3 does not affect the use of the watchband 10, the watchband 10 may fit the wrist, such that the blood pressure measuring unit 2 fits the user wrist to measure the user's blood pressure better.

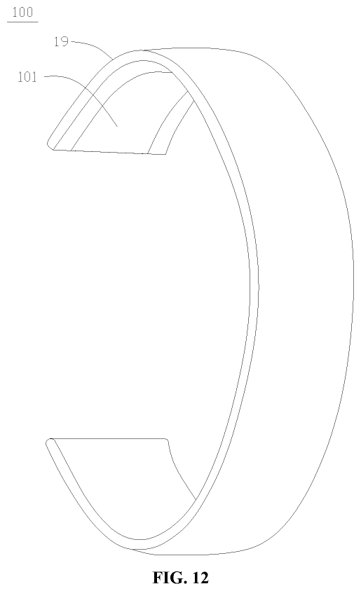

This embodiment further provides a wristband 100, which is suitably used on a wrist. Referring to FIG. 12, the wristband 100 includes a band body 19, and a circuit board 3 arranged on the band body 19 and a blood pressure measuring unit 2 arranged on the band body 19 and electrically connected with the circuit board 3 to measure a human blood pressure. Both ends of the band body 19 are detachably connected, that is, the wrist 100 in this embodiment may be used without cooperating with the watch head, to implement a connection and fit with the use on the wrist. Specifically, both ends of the band body 19 may be connected through the magic stickers 101, buckles and the like, or may also adopt a detachable structure and the like in a chain watchband.

Referring to FIGS. 1-10 in one possible implementation, the circuit board 3 is a flexible circuit board, which is arranged in the watchband 1. The flexible circuit board has soft toughness and can be bent to fit the use of the watch accessory 10, when the watch accessory 10 is fixed to a user wrist, the circuit board 3 does not affect the use of the watch accessory 10, the watch accessory 10 may fit the wrist, such that the blood pressure measuring unit 2 fits the user wrist to measure the user's blood pressure better.

To implement the blood pressure measurement by the blood pressure measuring unit 2, the blood pressure measuring unit 2 may adopt the following structure.

A first structure of the blood pressure measuring unit 2: the blood pressure measuring unit 2 includes a photoelectric sensor 24 that is arranged on the watchband 1 and electrically connected with the circuit board 3, when the watch accessory 10 is fixed to the wrist and measures the blood pressure, the photoelectric sensor 24 aligns with the artery on the wrist, an emitting light in the photoelectric sensor 24 irradiates the artery, a blood fluctuation in the artery caused by a human heart beat will cause the intensity of the feedback reflected light to change, resulting in different intensities of the light received by a receiving end in the photoelectric sensor 24, to generate a pulse wave electrical signal; and the pulse wave electrical signal is fed back to the circuit board 3, and the circuit board 3 calculates a blood pressure value according to the pulse wave electrical signal to measure the blood pressure. In an actual application of the structure, an inner side wall of the watchband 1 is provided with through holes or light-transmitting materials at both corresponding positions of an emitting end and a receiving end in the photoelectric sensor 24, so as not to affect the blood pressure measurement.

A second structure of the blood pressure measuring unit 2: the circuit board 3 is integrated with an MCU 34 and an air pump drive circuit 35 electrically connected with the MCU 34, the blood pressure measuring unit 2 includes an air pump 23 electrically connected with the air pump drive circuit 35, an air bag 21 connected with the air pump 23 and a pressure sensor 22 electrically connected with the MCU 33. The air pump 23 is arranged in the watchband 1, and the air bag 21 is arranged in a length direction of an inner side wall of the watchband 1. The air bag 21 applies a pressure to the user wrist when the air pump 23 is driven to inflate, the pressure sensor 22 is configured to detect a pressure value when the user's pulse beats, generate the pulse wave electrical signal and feed back the signal to the MCU 34, and the pressure sensor 22 is configured to detect a pressure value change during inflation and deflation of the air bag 21, generate an amplitude change electrical signal and feed back the signal to the MCU 22; and the MCU 22 calculates the blood pressure value according to the pulse wave electrical signal and the amplitude change electrical signal, to implement the blood pressure measurement.

The air pump 23 is arranged in the watchband 1, arranged together with the circuit board 3 in a thickness direction or a length direction and a width direction of the watchband 1.

In one possible implementation, the watch accessory 10 further includes a cell 5 and a wireless charging coil 6 that are electrically connected with the circuit board 3 and arranged in the watchband 1, the cell 5 is used for supplying power to the circuit board 3, the wireless charging coil 6 may be used in cooperation with a wireless charger of the existing smart watch on the market, and the cell 5 may be charged through the circuit board 3. Certainly, in other embodiments, the cell 5 may also be a disposable cell 5, which cannot be charged, etc. Alternatively, in the embodiment with the cell 5, the watch accessory 10 further includes a charging contact that is electrically connected with the circuit board 3, and the charging contact can also implement to charge the cell 5 when used in external connection with an adapter.

In one possible implementation, the watchband 1 includes a protective housing 13 and a band body 19 connected with the protective housing 13, and a mounting slot 131 for placing the watch head 00 is formed in the protective housing 13. The circuit board 3, the air pump 23 and the cell 5 are arranged on the band body 19 or the protective housing 13, and the air bag 21 is arranged in a length direction of the band body 19, as shown in FIG. 1 Thus, the watch head 00 is detachably mounted in the mounting slot to fix the watch head 00, specifically a side wall of the mounting slot 131 is a flexible glue structure, that is, when the watch head 00 is placed in the mounting slot 131, the watch head 00 may be wrapped and fixed, to facilitate the use when the user replaces the watch head 00. It is to be understood that the user may set the circuit board 3, the cell 5, the wireless charging coil 6 and the air pump 23 at the required position, and specific locations thereof are not defined herein.

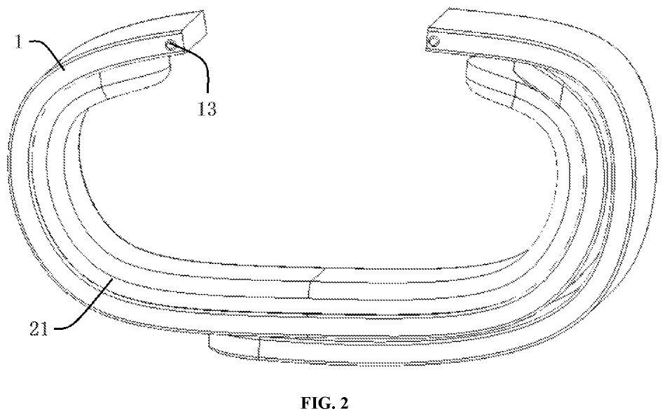

In other embodiments, as shown in FIG. 2, the the watchband 1 includes a band body 19, of which both ends are provided with connectors 13 detachably connected with the watch head 00. Specifically, both ends of the watchband 1 are detachably fixed to the watch head 00, to achieve a connection with the watch head 00 and a role of replacing the watch head 00.

In one possible implementation, a storage slot 14 for placing drugs is formed in the watchband 1, and a notch of the storage slot 14 is provided with a cover body 141 to place some small tablets, to facilitate the use when the user goes out. Specifically, the storage slot 14 may be arranged on the band body 19 or the protective housing 13.

In one possible implementation, the watch accessory 10 further includes a physiological index collecting unit 8 that is electrically connected with the circuit board 3 and configured to collect physiological indexes of a wearer in real time or periodically, and the physiological indexes include one or more of a blood pressure value, a heart rate value, blood oxygen, blood glucose, a body temperature, blood lipid, uric acid and a motion numerical value. One or more of the human heart rate value, blood oxygen, blood glucose, body temperature, blood lipid, uric acid and motion numerical value are collected through the physiological index collecting unit, and the functionality and the practical performance of the watch accessory 10 in this embodiment may be improved, to facilitate the use of the user.

In one possible implementation, the watch accessory 10 further includes a wireless transmission unit 7 that is electrically connected with the circuit board 3 and used for communicating with a mobile terminal, the circuit board 3 is configured to upload the blood pressure value, collected by the blood pressure measuring unit 2, and the physiological indexes, collected by the physiological index collecting unit 8, to a cloud 20 through the wireless transmission unit 7, and in cooperative use with the mobile terminal and the cloud 20, the monitoring may be implemented through the mobile terminal and the cloud 20. Specifically, the wireless transmission module 7 may be a radio frequency module, such as a Bluetooth module, a 2G module, a 3G module, a 4G module, a 5G module and a 2.4G module.

In the above implementations, the circuit board 3 may be in integrated injection molding with the watchband 1, or convexly arranged on the watchband 1. Alternatively, a position of the watchband 1 that corresponds to the circuit board 3, the blood pressure measuring unit 2, the wireless transmission unit 7, the physiological index collecting unit 8, the cell 5 and the wireless charging coil 6 is provided with a groove 15, the circuit board 3, the blood pressure measuring unit 2, the wireless transmission unit 7, the physiological index collecting unit 8, the cell 5 and the wireless charging coil 6 are placed in the respective fit groove 15, a glue filling layer for wrapping the circuit board 3, the blood pressure measuring unit 2, the wireless transmission unit 7, the physiological index collecting unit 8, the cell 5 and the wireless charging coil 6 in respective is arranged in the groove 15, and fixing the circuit board 3, the blood pressure measuring unit 2, the wireless transmission unit 7, the physiological index collecting unit 8, the cell 5 and the wireless charging coil 6 to the watchband 1 may also be implemented. In another embodiment, the watch accessory 10 further includes a housing 4 that is separately mounted with the circuit board 3, the blood pressure measuring unit 2, the wireless transmission unit 7, the physiological index collecting unit 8, the cell 5 and the wireless charging coil 6; the housing 4 may be arranged outside the watchband 1, or the housing 4 is arranged in the groove 15 and in interference fit with the groove 15 to fix the housing 4, and certainly, in other embodiments, the housing 4 may also be clamped in the groove 15 to fix the housing 4, etc.

Based on an enough thickness of the watch accessory 10, the flexible circuit board 3, the blood pressure measuring unit 2, the wireless transmission unit 7, the physiological index collecting unit 8, the cell 5 and the wireless charging coil 6 may be arranged up and down in a thickness direction of the watch accessory 10; alternatively, in order to reduce the thickness of the watch accessory 10, the blood pressure measuring unit 2, the wireless transmission unit 7, the physiological index collecting unit 8, the cell 5 and the wireless charging coil 6 may be arranged in a width direction or a length direction of the watch accessory 10, etc.

In an actual application, a wiring hole 41 that communicates with an interior and is used for wiring is formed in the housing 41, a plurality of wiring channels 17 are arranged on the watchband 1, and the plurality of wiring channels 17 are electrically connected lead wiring in respective among various components of the circuit board 3, the blood pressure measuring unit 2, the wireless transmission unit 7, the physiological index collecting unit 8, the cell 5 and the wireless charging coil 6, such that the circuit board 3 is electrically connected with the blood pressure measuring unit 2, the wireless transmission unit 7, the physiological index collecting unit 8, the cell 5 and the wireless charging coil 6 through the lead wiring.

In the embodiment that the blood pressure measuring unit 2 includes the air pump 23, the air bag 21 and the pressure sensor 22, the housing 4 used for mounting the air pump 23 is also provided with an air outlet 151 and an air inlet 152 that are connected with the air pump 23 through an air pipe 231, to facilitate the connection between the air pump 23 and the air bag 21.

In one possible implementation, the physiological index collecting unit 8 includes a fall sensor 81 that is electrically connected with the circuit board 3 and arranged in the watchband 1. The circuit board 3 judges whether the user falls or not according to the electrical signal fed back by the fall sensor 81, when the circuit board 3 judges that the user falls, position information is uploaded to the cloud 20 through the wireless transmission unit 7, the cloud 20 may inform the user in matching use, such that falling information of the user wearing the watch accessory 10 in this embodiment is informed to the user at the monitoring end, and then the user at the monitoring end can make a response.

In one possible implementation, the watch accessory 10 further includes an alarm module 31 that is electrically connected with the circuit board 3 and arranged on the watchband 1, and the alarm module 31 is configured to send an alarm signal when the circuit board 3 judges that the user falls to inform the people around. The alarm module 31 is configured to inform the user wearing the watch accessory 10 when the physiological indexes of the circuit board 3 exceed a threshold value, such that the user can see a doctor in time, to avoid a delay.

In one possible implementation, the watch accessory 10 further includes a positioning unit 32 that is electrically connected with circuit board 3 and used for detecting a user position, and the positioning unit 32 is arranged in the watchband 1. When the circuit board 3 judges that the user falls, the position information positioned by the positioning unit 32 is uploaded to the terminal through the wireless transmission unit 7, such that the user at the monitoring end can know the specific position of the user wearing the watch accessory 10 and make a response in time. The positioning unit 32 may be a GPS (Global Positioning System) sensor, etc.

Specifically, in one possible implementation, the physiological index collecting unit 8 includes an acceleration sensor 82 that is electrically connected with the circuit board 3, the acceleration sensor 82 is arranged in the watchband 1, the circuit board 3 calculates a motion numerical value according to an electrical signal fed back by the acceleration sensor 82, and the motion numerical value is a step number, a motion distance, a calorie consumption and the like, such that the user can know about the motion numerical value.

In one possible implementation, the watch accessory 10 further includes a heart rate sensor 83 that is electrically connected with the circuit board 3 and arranged in the watchband 1, and the circuit board 3 calculates a heart rate value according to an electrical signal fed back by the heart rate sensor 83, such that the users can know about their own heart rate.

In one possible implementation, the watch accessory 10 further includes a display 18 that is electrically connected with the circuit board 3, the display 18 is configured to display the physiological indexes and the testing numerical value, and the testing numerical value may be the position information and the like.

The watch accessory 10 further includes a switching element 33 that is electrically connected with the circuit board 3 and arranged on the watchband 1, the circuit board 3 is configured to receive an electrical signal through which the user operates the switching element 33 and/or receive an electrical signal for operation at an APP (Application) end, and start a detection function or switch the physiological indexes displayed on the display 18.

In one possible implementation, the watchband 1 is adjusted in length to be suitably used for wrist widths of different users, and the applicability of the watch accessory 10 is improved.

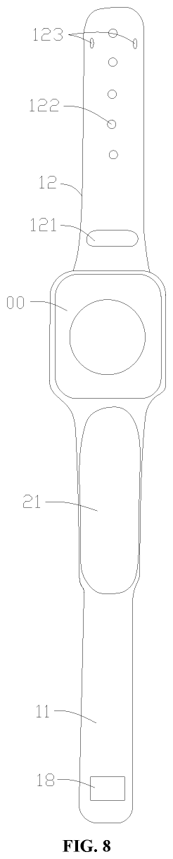

Specifically, referring to FIGS. 8-11, the band body 9 includes an upper watchband 12 and a lower watchband 11, each of which has one end detachably connected with the watch head 00, the air bag 21 is arranged at one end of a lower surface of the lower watchband 1 that is close to the watch head 00, the air pump 23 and the pressure sensor 22 are arranged on the lower watchband 1 and close to one end of the watch head 00, and the air pump 23 is located on one side of the pressure sensor 22 that is away from the air bag 21. Both the physiological index collecting unit 8 and the wireless transmission unit 7 are arranged on the circuit board 3, the circuit board 3 is located on the lower watchband 1 and located at one end of the air bag 21, both the cell 5 and the display screen 18 are located at one end of the lower watchband 1 that is away from the watch head 00, and the display screen 18 and the air bag 21 are located on the same side.

A threading hole 121 is formed in one end of the upper watchband 12 that is close to the watch head 00, one end of the lower surface of the upper watchband 12 that is away from the watch head 00 is provided with a second block 123, and a plurality of second card slots 113 extending in a length direction of the watchband 1 are formed at a position where the lower watchband 11 corresponds to the air bag 21. When the watchband 1 is worn on a wrist and after the lower watchband 11 passes through the threading hole 121 at one end away from the watch head 00, the second block 123 is placed in the second card slot 113 that is in the direction close to the watch head 00, and after folded back and forth at one end away from the watch head 00, the lower watchband 11 is detachably connected with the upper watchband 12, to achieve the adjustable wearing length of the watchband 1 when the circuit board 3, the air bag 21, the air pump 23 and the pressure sensor 22 are reasonably arranged, thereby being suitable for different wrists.

A plurality of third card slots 123 extending in the length direction of the watchband 1 are formed in the upper watchband 12, a third block 114 is arranged at a position where the lower surface of the lower watchband 11 is away from the watch head 00, and after folded back and forth at one end of the lower watchband 11, the third block 114 is clamped with the third card slots 123, such that the lower watchband 11 is fixedly connected with the upper watchband 12.

Certainly, in other implementations, after folded back and forth at one end away from the watch head 00, the lower watchband 11 is fixed together with the upper watchband 11 through magnet adsorption, magic stickers, magnetic buckles, etc.

The lower watchband 11 includes a first watch section 111 detachably connected with the watch head 00 and a second watch section 112 connected with the first watch section 111, the air bag 21 is located on the first watch section 111 or the second watch section 112, the circuit board 3 is at least partially located on the first watch section 111, in this embodiment, the air bag 21, the air pump 23, the pressure sensor 22 and the second card slots 113 are all located on the first watch section 111, a length of the second watch section 111 may be equal to that of the upper watchband 12 but is not limited to the above, a width of the threading hole 121 is greater than that of the second watch section 112 but less than that of the first watch section 111, to achieve the stable wearing of the watchband 1 when the circuit board 3, the air bag 21, the air pump 23 and the pressure sensor 22 are arranged on the band body 19.

The length of the air bag 21 may be 8-15 cm (but is not limited to this), the second card slot 113 is in a two-column setting and close to both sides of the first watch section 111, such that the watchband 1 fits with different wrists. Specifically, the length of the air bag 21 is 13 cm.

In an actual application, the upper watchband 12 and the lower watchband 11 may not be connected with the watch head 00, and in this state, two opposite ends of the upper watchband 12 and the lower watchband 11 may be detachably connected, that is, the wristband 100 is formed.

This embodiment further provides a user monitoring and management system used in cooperation with the foregoing watch accessory 10, to facilitate the use of the user. For example, the watch accessory 10 may be worn by elderly people living alone, such that children living far away can know about the physiological indexes of the parents, to play a role in monitoring the heath of the watch accessory 10 user, timely response can be made when anomalies are found, and the heath management of the elderly people living alone is improved.

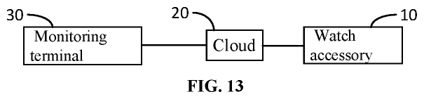

Referring to FIG. 13, the user monitoring and management system includes the watch accessory 10 and the cloud 20 communicating with the watch accessory 10 that are described above, the cloud 20 is configured to receive physiological indexes transmitted by the wireless transmission unit 7 and perform monitoring, and to send a first alarm signal to the wireless transmission unit 7 when the physiological indexes exceed a threshold value; and the circuit board 3 receives the first alarm signal fed back by the cloud 20 through the wireless transmission unit 7 and sends an alarm through an alarm module 31, such that the watch accessory 10 users can know about their own physical conditions and make an appointment with a doctor in time for treatment. Specifically, the alarm module 31 may be an indicator light or a horn and the like electrically connected with the circuit board 3.

In one possible implementation, the user monitoring and management system further includes a monitoring terminal 30 that communicates with the cloud 20. The cloud 20 is also configured to send a second alarm signal to the monitoring terminal 30 when the physiological indexes exceed the threshold value during a preset time period, and the monitoring terminal 30 receives a reminding signal sent by the second alarm signal; and specifically the monitoring terminal 30 is a mobile phone, a computer, a smart watch and the like at the monitoring end, such that an alarm is sent when the watch accessory 10 user has the abnormal physiological indexes, to play reminding and preventable roles. The cloud 20 may be APP, the cloud 20 is further configured to transmit an execution measurement signal to the watch accessory 10 during user operation, that is, the user performs an operation on the APP to transmit the execution measurement signal to the watch accessory 10, and the circuit board 3 of the watch accessory 10 receives the execution measurement signal through the wireless transmission unit 7 to drive the blood pressure measuring unit 2 to work, to facilitate the user's operation and use at the terminal.

It should be noted that, in this embodiment of the present disclosure, all directional indications (such as up, down, left, right, front, and back). It is only used to explain a relative positional relationship, a motion condition, and the like between components in a specific posture (as shown in the accompanying drawings). If the specific posture changes, the directional indication changes accordingly.

In addition, descriptions in the present disclosure relating to “first”, “second”, and the like are used only for description purposes, and cannot be understood to indicate or imply relative importance or implicitly indicate a quantity of indicated technical features. Therefore, a feature defined with “first” and “second” may explicitly or implicitly include at least one feature. In addition, the “and/or” in this specification includes three solutions, using an and/or B as an example, including a technical solution A, a technical solution B, and a technical solution that both an and B meet. In addition, the technical solutions between the embodiments may be combined with each other. However, a person of ordinary skill in the art may be used as a basis for implementation. When a combination of the technical solutions is contradictory or cannot be implemented, it should be considered that a combination of the technical solutions does not exist, and is not within the protection scope required by the present disclosure.

The foregoing descriptions are merely preferred embodiments of the present disclosure, and are not intended to limit the patent scope of the present disclosure. Any equivalent structural transformation performed by using the content in the specification and accompanying drawings of the present disclosure or directly/indirectly applied in another related technical field is included in the protection scope of the present disclosure.

1. A watch accessory, comprising:
a watchband detachably connected with a watch head;
a circuit board arranged on the watchband;
a blood pressure measuring unit arranged on the watchband and electrically connected with the circuit board to measure a human blood pressure.

2. The watch accessory according to claim 1, wherein the circuit board is a flexible circuit board, which is arranged in the watchband.

3. The watch accessory according to claim 1, wherein the blood pressure measuring unit comprises a photoelectric sensor that is arranged on the watchband and electrically connected with the circuit board; and
the photoelectric sensor generates a pulse wave electrical signal according to an intensity of a reflected light fed back by a blood fluctuation due to a human heart beat, the pulse wave electrical signal is fed back to the circuit board, and the circuit board calculates a blood pressure value according to the pulse wave electrical signal.

4. The watch accessory according to claim 1, wherein the blood pressure measuring unit comprises an air pump electrically connected with the circuit board, an air bag connected with the air pump and a pressure sensor electrically connected with the circuit board, the air pump is arranged in the watchband, and the air bag is arranged in a length direction of an inner side wall of the watchband; and
the air bag applies a pressure to a user wrist when the air pump is driven to inflate, the pressure sensor is configured to detect a pressure value when the user's pulse beats, generate the pulse wave electrical signal, and detect an amplitude change electrical signal generated due to a pressure value change during an inflating and deflating process of the air bag; and the circuit board calculates the blood pressure value according to the pulse wave electrical signal and the amplitude change electrical signal.

5. The watch accessory according to claim 4, further comprising a cell and a wireless charging coil that are electrically connected with the circuit board and arranged in the watchband, and the cell is used for supplying power to the circuit board.

6. The watch accessory according to claim 5, wherein the watchband comprises a protective housing and a band body connected with the protective housing, and a mounting slot for placing the watch head is formed in the protective housing; and
the circuit board, the air pump and the cell are arranged on the band body or the protective housing, and the air bag is arranged in a length direction of the band body.

7. The watch accessory according to claim 5, wherein the watchband comprises a band body, both ends of the band body are provided with connectors detachably connected with the watch head, the air bag is arranged in a length direction of the band body, and a length of the air bag is in a range from 8-15 cm.

8. The watch accessory according to claim 7, further comprising a physiological index collecting unit that is electrically connected with the circuit board, the physiological index collecting unit is configured to collect physiological indexes of a wearer in real time or periodically, and the physiological indexes comprise one or more of a blood pressure value, a heart rate value, blood oxygen, blood glucose, a body temperature, blood lipid, uric acid and a motion numerical value, the watch accessory further comprises a display screen disposed on the band body, and both the air bag and the display screen are located at the same side of the band body.

9. The watch accessory according to claim 8, further comprising a wireless transmission unit that is electrically connected with the circuit board and used for communicating with a mobile terminal, the circuit board is configured to upload the blood pressure value, collected by the blood pressure measuring unit, and the physiological indexes, collected by the physiological index collecting unit, to a cloud through the wireless transmission unit.

10. The watch accessory according to claim 9, further comprising a housing that is mounted with the circuit board, the blood pressure measuring unit, the wireless transmission unit, the physiological index collecting unit, the cell and the wireless charging coil; and
the housing is arranged outside the band body, or a groove is formed on or inside the band body, and the housing is arranged in the groove and in interference fit with the groove.

11. The watch accessory according to claim 10, wherein wiring holes that communicate with an interior of the housing are formed in the housing, a plurality of wiring channels are arranged on the band body, and a plurality of wires passing through the plurality of wiring channels and the wiring holes are electrically connected between the circuit board, the blood pressure measuring unit, the wireless transmission unit, the physiological index collecting unit, the cell and the wireless charging coil; and
the blood pressure measuring unit comprises the air pump electrically connected with the circuit board, the air bag connected with the air pump and the pressure sensor electrically connected with the circuit board, and the housing used for mounting the air pump is also provided with an air outlet and an air inlet that are connected with the air pump.

12. The watch accessory according to claim 9, wherein the physiological index collecting unit comprises a fall sensor that is electrically connected with the circuit board and arranged in the band body, and the circuit board judges whether the user falls or not according to an electrical signal fed back by the fall sensor; and
when the circuit board judges that the user falls, a falling information is uploaded to the cloud through the wireless transmission unit.

13. The watch accessory according to claim 12, further comprising an alarm module that is electrically connected with the circuit board and arranged on the band body, the alarm module is configured to send an alarm signal when the circuit board judges that the user falls or the physiological indexes exceed a threshold value.

14. The watch accessory according to claim 13, further comprising a positioning unit that is electrically connected with the circuit board and used for detecting a user position, and the positioning unit is arranged in the band body; and
when the circuit board judges that the user falls, a position information positioned detected by the positioning unit is uploaded to the terminal through the wireless transmission unit.

15. The watch accessory according to claim 9, wherein the physiological index collecting unit comprises an acceleration sensor that is electrically connected with the circuit board, the acceleration sensor is arranged in the band body, the circuit board calculates a motion numerical value according to an electrical signal fed back by the acceleration sensor, and the motion numerical value is one or more of a step number, a motion distance and a calorie consumption.

16. The watch accessory according to claim 9, further comprising a heart rate sensor that is electrically connected with the circuit board and arranged in the band body, and the circuit board calculates a heart rate value according to an electrical signal fed back by the heart rate sensor.

17. The watch accessory according to claim 9, further comprising a display that is electrically connected with the circuit board, and the display is configured to display the physiological indexes; and
the band body further comprises a switching element that is electrically connected with the circuit board and arranged on the band body, the circuit board is configured to receive an electrical signal through which the user operates the switching element and/or receive an electrical signal for operation at an APP (Application) end, and start a detection function or switch the physiological indexes displayed on the display.

18. The watch accessory according to claim 9, wherein
the band body comprises an upper watchband and a lower watchband, each of which has one end detachably connected with the watch head, the air bag is arranged at one end of a lower surface of the lower watchband that is close to the watch head, the air pump and the pressure sensor are arranged on the lower watchband and close to one end of the watch head;
both the physiological index collecting unit and the wireless transmission unit are arranged on the circuit board, both the cell and the display screen are located at one end of the lower watchband that is away from the watch head, and the display screen and the air bag are located on the same side;
a threading hole is formed in one end of the upper watchband that is close to the watch head, one end of the lower surface of the upper watchband that is away from the watch head is provided with a second block, and a plurality of second card slots extending in a length direction of the watchband are formed at a position where the lower watchband corresponds to the air bag; and
when the watchband is worn on a wrist and after the lower watchband passes through the threading hole at one end away from the watch head, the second block is placed in the second card slot that is in the direction close to the watch head, and after folded back and forth at one end away from the watch head, the lower watchband is detachably connected with the upper watchband.

19. The watch accessory according to claim 18, wherein a plurality of third card slots extending in the length direction of the watchband are formed in the upper watchband, a third block is arranged at a position where the lower surface of the lower watchband is away from the watch head, and after folded back and forth at one end of the lower watchband, the third block is clamped with the third card slots.

20. The watch accessory according to claim 18, wherein after folded back and forth at one end away from the watch head, the lower watchband is fixed together with the upper watchband through magnet adsorption or magic stickers.

21. The watch accessory according to claim 18, wherein the lower watchband comprises a first watch section detachably connected with the watch head and a second watch section connected with the first watch section, the air bag is located on the first watch section or the second watch section, the circuit board is at least partially located on the first watch section, and a width of the threading hole is greater than that of the second watch section but less than that of the first watch section.

22. The watch accessory according to claim 21, wherein the second card slot is in a two-column setting and close to both sides of the first watch section.

23. The watchband according to claim 1, wherein a storage slot for placing drugs is formed in the watchband, and a notch of the storage slot is provided with a cover body, and wherein the watchband comprises an inner surface, an outer surface opposite to the inner surface and a side surface connected between the inner surface and the outer surface, the notch of the storage slot is located at the side surface and located at an end of the watchband adjacent to the watch head.

24. A wristband, comprising:
a band body having two ends detachably connected;
a circuit board arranged on the band body; and
a blood pressure measuring unit arranged on the band body and connected with the circuit board to measure a human pressure blood.

25. A user monitoring and management system, comprising:
a watch accessory according to claim 9;
the cloud that communicates with the watch accessory, wherein the cloud is configured to receive physiological indexes transmitted by the wireless transmission unit and perform monitoring, and to send a first alarm signal to the wireless transmission unit when the physiological indexes exceed a threshold value; and
the circuit board receives the first alarm signal fed back by the cloud through the wireless transmission unit and sends an alarm through an alarm module.

26. The user monitoring and management system according to claim 25, further comprising a monitoring terminal that communicates with the cloud;
the cloud is also configured to send a second alarm signal to the monitoring terminal when the physiological indexes exceed the threshold value during a preset time period, and the monitoring terminal is configured to receives the second alarm signal and output a reminding signal; and
the cloud is further configured to transmit an execution measurement signal to the watch accessory during user operation, and the circuit board receives the execution measurement signal through the wireless transmission unit to drive a blood pressure measuring unit to work.
